(19) United States
(12) Patent Application Publication
(10) Pub. No.: US 2006/0020209 A1
(43) Pub. Date: Jan. 26, 2006

(54) Ultrasonic imaging apparatus

(75) Inventor: Hiroshi Hashimoto, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/169,369

(22) Filed: Jun. 29, 2005

(30) Foreign Application Priority Data
Jul. 6, 2004 (JP) 2004-199417

(51) Int. Cl. A61B 8/14

(52) U.S. Cl. 600/458

(57) ABSTRACT

A method for reducing a burden on an operator at the time of administration of a contrast agent and thereby performing a stable contrast inspection, a time up to a contrast-agent administration start time is counted down by a contrast timer. The countdown is notified by a voice output unit via voice. On the basis of the voice-based countdown, for example, the operator administers the contrast agent to a subject at the contrast-agent administration start time. An ultrasonic sound is scanned over a region of the subject including a region of interest over which the contrast agent is distributed, so that echoes are received from the scanned region of subject. The scan of the ultrasonic probe and the generation of an image by image generating device are carried out during a predetermined time. The time required to perform the contrast inspection is measured by the contrast timer.

### BACKGROUND OF THE INVENTION

The present invention relates to an ultrasonic imaging apparatus, and particularly to an ultrasonic imaging apparatus which effects ultrasonic photography on a subject with a contrast agent administered thereto.

In ultrasound photography or imaging, a tomogram is photographed or imaged using echoes of ultrasounds transmitted to the interior of a subject. The photographed tomogram is displayed as a B-mode image or a harmonic image. A dynamic image such as a blood flow or the like is photographed using an ultrasonic Doppler shift and displayed as a color Doppler image.

When it is necessary to enhance an echo intensity, a contrast agent is distributed over a region of interest (ROI) using the blood flow. The contrast agent is a collection of small foam whose diameter is about a few μm. An inspection using the contrast agent has been mentioned in a patent document 1.

The time required to cause the contrast agent to be distributed over the region of interest upon the contrast inspection becomes a time interval very important for diagnosis. That is, 2 to 30 seconds counted from immediately after the administration of the contrast agent become a time zone very important upon an ultrasonic photography or imaging inspection using the contrast agent.

A contrast clock indicative of a time interval from timing for administration has heretofore been used upon the contrast inspection. The administration of the contrast agent is started simultaneously when an operation or control button for the contrast clock is pressed.

[Patent Document 1] Japanese Unexamined Patent Publication No. 2004-147823

Upon the contrast inspection, one person administers a contrast agent and another person causes an ultrasonic sound to scan over a subject, i.e., an ultrasonic probe is brought into contact with the subject. Then, the two persons call to each other and the scanning person turns on a contrast timer and at the same time other person administers the contrast agent to the subject.

A problem arises in that since the control button for the contrast timer is placed on an ultrasonic diagnostic apparatus, a scanned region for the subject is shifted because the button is pressed. Therefore, it is necessary to prepare a person for simply pressing the contrast timer, thus causing a problem that operationality is poor.

In order to provide a stable contrast inspection, there is a need to reduce a burden on an operator at the time of administration of a contrast agent and allow the operator to concentrate on a scan made to a subject upon administration of the contrast agent and on an observation of an image reflected on a monitor by the scan.

### SUMMARY OF THE INVENTION

Therefore, an object of the present invention is to provide an ultrasonic imaging apparatus which reduces a burden on an operator at the time of administration of a contrast agent to thereby make it possible to carry out a stable contrast inspection.

In order to achieve the above object, there is provided an ultrasonic imaging apparatus of the present invention, which comprises an ultrasonic transmit-receive means which scans an interior of a subject by ultrasounds and thereby receives echoes, an image generating means which generates an image based on the received echoes, a time measuring means which counts down a time from a pre-set time to the time when the administration of a contrast agent is started, and a notifying means which notifies the time up to the contrast-agent administration start time, counted down by the time measuring means.

In the ultrasonic imaging apparatus of the present invention, the time measuring means counts down the time up to the contrast-agent administration start time, and the notifying means notifies the counted-down time up to the contrast-agent administration start time.

On the basis of the countdown measured by the notifying means, for example, an operator administers the contrast agent to the subject. An ultrasonic sound is scanned over a subject's region including a region of interest over which the contrast agent is distributed, by the ultrasonic transmit-receive means, so that echoes are received from the scanned region of the subject. Then, an image is generated based on the received echoes by the image generating means.

The scan of the ultrasonic transmit-receive means and the generation of the image by the image generating means are carried out during a predetermined time. This time is measured by the time measuring means.

In order to achieve the above object, there is provided an ultrasonic imaging apparatus of the present invention, which comprises an ultrasonic transmit-receive means which scans an interior of a subject by ultrasounds and thereby receives echoes from the subject, an image generating means which generates an image based on the received echoes, a time measuring means which counts down a time from a pre-set time to the time when the administration of a contrast agent is started, and a contrast-agent administering means which administers a contrast agent to the subject, based on countdown information obtained by the time measuring means when the contrast-agent administration start time is reached.

In the ultrasonic imaging apparatus of the present invention, the time measuring means counts down the time up to the contrast-agent administration start time, and the contrast-agent administering means administers the contrast agent to the subject on the basis of the counted-down information when the contrast-agent administration start time is reached.

When the contrast agent is administered, an ultrasonic sound is scanned over a subject's region including a region of interest over which the contrast agent is distributed, by the ultrasonic transmit-receive means, so that echoes from the scanned region of subject are received. Then, the image generating means generates an image, based on the received echoes.

The scan of the ultrasonic transmit-receive means and the generation of the image by the image generating means are carried out during a predetermined time. This time is measured by the time measuring means.

According to the present invention, a burden on an operator at the time of administration of a contrast agent is reduced, thereby making it possible to carry out a stable contrast inspection.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing one example of a configuration of an ultrasonic imaging apparatus according to first and second embodiments.

FIG. 2 is a block diagram illustrating one example of a configuration of a transmit-receive section.

FIG. 3 is a block diagram depicting one example of a configuration of a B mode processor.

FIG. 4 is a block diagram showing one example of a configuration of a Doppler processor.

FIG. 5 is a block diagram illustrating one example of a configuration of an image processor.

FIG. 6 is a flowchart showing a procedure for a contrast inspection.

FIG. 7 is a diagram showing the manner of the contrast inspection.

FIGS. 8(a) through 8(c) are respectively diagrams showing countdown notified images by a display unit.

FIG. 9 is a block diagram illustrating one example of a configuration of an ultrasonic imaging apparatus according to a third embodiment.

### DETAILED DESCRIPTION OF THE INVENTION

Embodiments showing ultrasonic imaging apparatuses of the present invention will hereinafter be described with reference to the accompanying drawings.

### First Embodiment

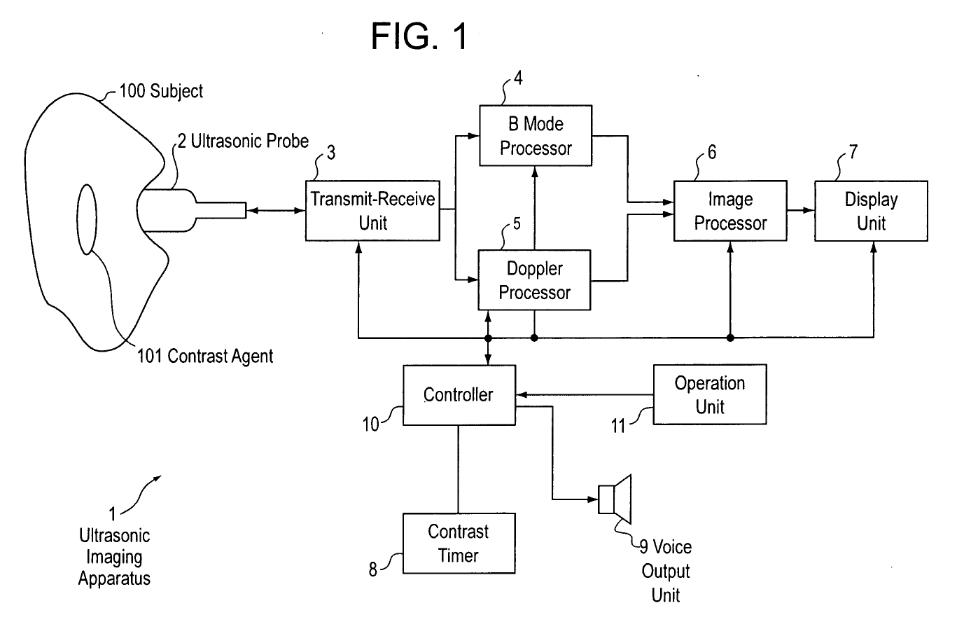

FIG. 1 is a block diagram showing one example of a configuration of an ultrasonic imaging apparatus according to the present embodiment.

The ultrasonic imaging apparatus according to the present embodiment has an ultrasound probe 2, a transmit-receive unit 3, a B mode processor 4, a Doppler processor 5, an image processor 6, a display unit 7, a contrast timer 8, a voice output unit 9, a controller 10 and an operation unit 11.

The ultrasonic probe 2 has an array of a plurality of ultrasonic transducers unillustrated in the drawing. The individual ultrasonic transducers are formed of a piezoelectric material such as PZT (lead zirconate (Zr) titanate (Ti)) ceramics or the like. The ultrasonic probe 2 is used in contact with a subject 100 by an operator. A contrast agent 101 is supplied to a region of interest through the use of a blood flow.

The ultrasonic probe 2 is connected to the transmit-receive unit 3. The transmit-receive unit 3 supplies a drive signal to the ultrasonic probe 2 to send an ultrasound wave. The transmit-receive unit 3 receives an echo signal received by the ultrasonic probe 2.

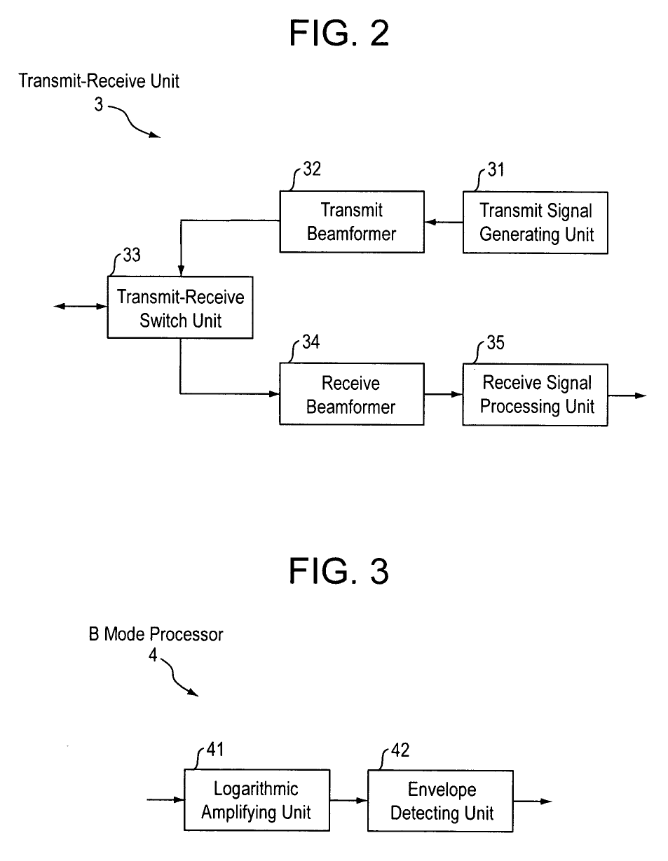

FIG. 2 is a block diagram showing one example of a configuration of the transmit-receive unit 3.

As shown in FIG. 2, the transmit-receive unit 3 has a transmit signal generating unit 31, a transmit beamformer 32, a transmit-receive switch unit 33, a receive beamformer 34, and a receive signal processing unit 35.

The transmit signal generating unit 31 periodically generates transmit signals and inputs the same to the transmit beamformer 32. The period of each transmit signal is controlled by the controller 10. The transmit signal generating unit 31 performs switching between ultrasonic scan modes in the vicinity of the start time of a contrast agent under the control of the controller 10. For example, transmit signals for generating ultrasounds identical in phase, for generating a B mode image are generated in the vicinity of the start time of the contrast agent. Transmit signals for alternately generating antiphase ultrasounds for generating a harmonic image are generated after the start time of the contrast agent.

The transmit beamformer 32 is used to perform beamforming for wave sending or transmission and generates a beamforming signal for forming an ultrasonic beam of a predetermined orientation, based on each transmit signal. The beamforming signal comprises a plurality of drive signals each added with a time difference associated with the orientation. The beamforming is controlled by the controller 10. The transmit beamformer 32 outputs the transmit beamforming signal to the transmit-receive switch unit 33.

The transmit-receive switch unit 33 outputs the beamforming signal to its corresponding ultrasonic transducer array. In the ultrasonic transducer array, the plurality of ultrasonic transducers that constitute a transmit aperture, respectively generates ultrasounds each having a phase difference corresponding to the difference in time between the drive signals. An ultrasonic beam along sound rays oriented in a predetermined orientation is formed by combining wavefronts of those ultrasounds.

The receive beamformer 34 is connected to the transmit-receive switch unit 33. The transmit-receive switch unit 33 outputs a plurality of echo signals received by a receive aperture in the ultrasonic transducer array to the receive beamformer 34.

The receive beamformer 34 is used to perform receive beamforming corresponding to transmit sound rays. The receive beamformer 34 applies time differences to a plurality of receive echoes to adjust phases and then adds them to thereby generate echo receive signals along sound rays oriented in a predetermined orientation. The receive beamforming is controlled by the controller 10.

The receive signal processing unit 35 extracts a secondary harmonic echo from the echo receive signals in a harmonic B mode. Although a basic wave echo is received from a subject upon generation of the B mode image, there is a need to receive a secondary harmonic echo from the contrast agent upon generation of the harmonic image. Therefore, echo signals from the subject, which have been obtained by two ultrasounds reversed in phase, are added together to thereby cancel out basic wave components, whereby only secondary harmonic components are enhanced and extracted.

The transmission of the ultrasonic beam is repeatedly performed at predetermined time intervals according to the transmit signals generated by the transmit signal generating unit 31. The transmit beamformer 32 and the receive beamformer 34 change the orientations of sound rays by predetermined amounts in accordance with the repetitive transmission. Consequently, the interior of the subject 100 is sequentially scanned according to the sound rays. The transmit-receive unit 3 performs so-called sector, linear and convex scans, etc.

Such scans are continuously performed under the control of the controller 10. The ultrasonic probe 2, the transmit-receive unit 3 and the controller 10 show one example of an embodiment of ultrasonic transmit-receive means of the present invention.

The transmit-receive unit 3 is connected to the B mode processor 4 and the Doppler processor 5. An echo signal set for each sound ray, which is outputted from the transmit-receive unit 3, is inputted to the B mode processor 4 and the Doppler processor 5.

The B mode processor 4 generates B-mode image data on the basis of a basic-wave echo receive signal or generates harmonic image data on the basis of a secondary harmonic receive signal. The harmonic image data is B mode image data generated based on the secondary harmonic receive signal from the contrast agent. FIG. 3 is a block diagram showing one example of a configuration of the B mode processor 4. The B mode processor 4 has a logarithmic amplifying unit 41 and an envelope detection unit 42.

In the B mode processor 4, the logarithmic amplifying unit 41 logarithmically amplifies each echo receive signal, and the envelope detection unit 42 detects an envelope thereof to obtain a signal indicative of the intensity of an echo at each reflecting point on a sound ray, i.e., an A scope signal, thereby forming B-mode image data or harmonic image data with respective instantaneous amplitudes of the A scope signal as luminance values respectively.

The Doppler processor 5 is used to form Doppler image data. The Doppler image data includes flow velocity data, distributed data and power data to be described later.

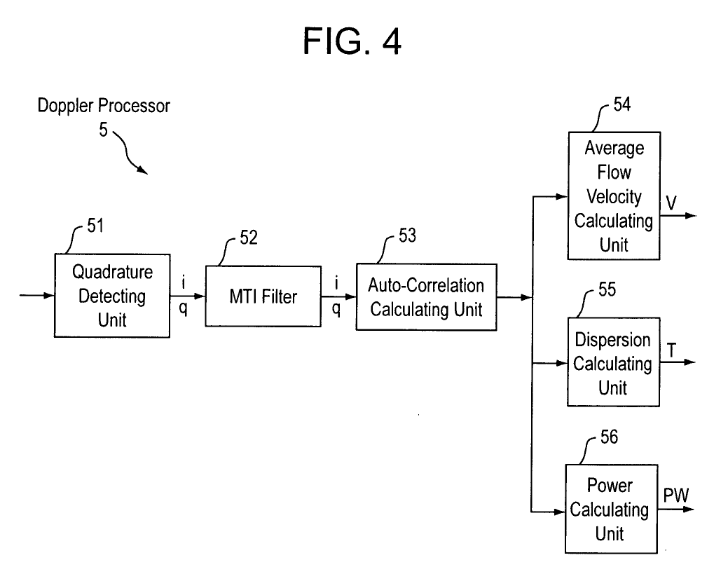

FIG. 4 is a block diagram showing one example of a configuration of the Doppler processor 5. As shown in FIG. 4, the Doppler processor 5 includes a quadrature detection unit 51, an MTI filter (Moving target indication filter) 52, an auto-correlation calculating unit 53, an average flow velocity calculating unit 54, a dispersion calculating unit 55 and a power calculating unit 56.

The Doppler processor 5 effects quadrature detection on each echo receive signal through the use of the quadrature detection unit 51 and performs MTI processing thereof through the use of the MTI filter 52 to thereby obtain a Doppler shift of each echo signal. Further, the Doppler processor 5 effects an auto-correlation calculation on a signal outputted from the MTI filter 52, through the use of the auto-correlation calculating unit 53. The Doppler processor 5 determines an average flow velocity V from the result of auto-correlation calculation through the use of the average flow velocity calculating unit 54. The Doppler processor 5 determines a dispersion T of a flow velocity from the result of auto-correlation calculation through the use of the dispersion calculating unit 55. Further, the Doppler processor 5 determines power PW of the Doppler signal from the result of auto-correlation calculation through the use of the power calculating unit 56. The average flow velocity is hereinafter also called simply a flow velocity. Further, the dispersion of the flow velocity is also simply called dispersion, and the power of the Doppler signal is also simply called power.

Respective data indicative of the flow velocity V, dispersion T and power PW of an echo source moved within the subject 100 are obtained for every sound ray by the Doppler processor 5. These data indicate the flow velocity, dispersion and power of each of pixels on sound rays. Incidentally, the flow velocity is obtained as a component lying in the direction of each sound ray. A distinction is made between the direction in which the subject approaches the ultrasonic probe 2 and the direction in which it moves away therefrom.

The B mode processor 4 and the Doppler processor 5 are connected to the image processor 6. The image processor 6 produces a B-mode image, a harmonic image and a Doppler image respectively, based on data respectively inputted from the B mode processor 4 and the Doppler processor 5. The B mode processor 4, the Doppler processor 5 and the image processor 6 show one example of an embodiment of image generating means of the present invention.

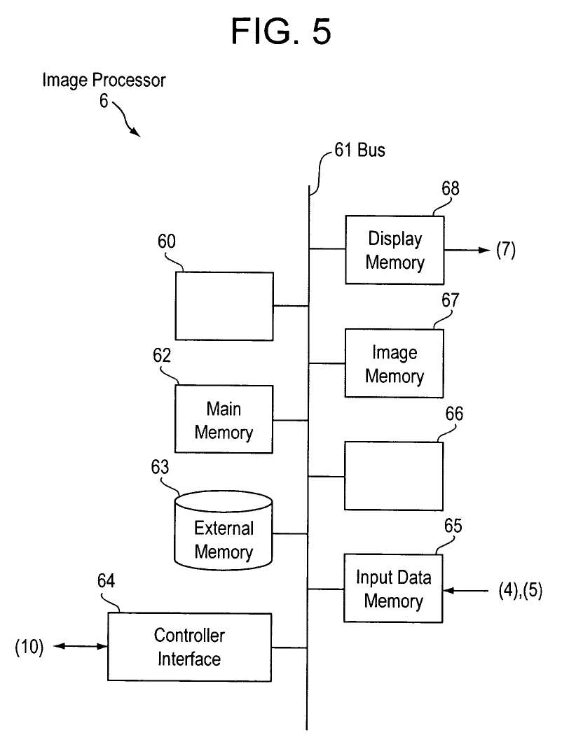

FIG. 5 is a block diagram showing one example of a configuration of the image processor 6.

As shown in FIG. 5, the image processor 6 has a central processing unit (CPU: Central Processing Unit) 60. A main memory 62, an external memory 63, a controller interface 64, an input data memory 65, a digital scan converter (DSC: Digital Scan Converter) 66, an image memory 67 and a display memory 68 are connected to a CPU 60 by a bus 61.

Programs to be executed by the CPU 60 are stored in the external memory 63. Various data used upon the execution of each program by the CPU 60 are also stored in the external memory 63.

The CPU 60 loads the corresponding program into the main memory 62 through the external memory 63 and executes it, thereby performing predetermined image processing. The CPU 60 performs the transmission and reception of control signals to and from the controller 10 through the controller interface 64.

B-mode image data, harmonic image data and Doppler image data inputted for each sound ray from the B mode processor 4 and the Doppler processor 5 are respectively stored in the input data memory 65. The data stored in the input data memory 65 are scanned and converted by the DSC 66 and stored in the image memory 67. The data of the image memory 67 are outputted to the display unit 7 through the display memory 68.

The display unit 7 is connected to the image processor 6. The display unit 7 is supplied with an image signal from the image processor 6 and displays an image, based on the image signal. The display unit 7 comprises a CRT or a liquid crystal display or the like capable of displaying a color image thereon.

The contrast timer 8 performs counting (countdown) ahead of a predetermined time alone and thereafter measures a time interval that has elapsed from 0 second. 0 second set as a base time corresponds to the time when the administration of a contrast agent is started. The time required to perform countdown can be set through the operation unit 11. The time is set to, for example, 10 seconds.

The voice output unit 9 is controlled by the controller 10 and notifies the countdown made by the contrast timer 8 via voice. The voice output unit 9 notifies the countdown of the contrast timer 8 via voice for each second as it is, for example. In addition to it, the countdown may also be notified for each second ahead of 5 seconds, for example. Incidentally, the notification made for each second may rely on reading of numerical values or rhythmic sound in one second cycle. The setting of these is carried out via the operation unit 11 in advance.

The controller 10 is connected to the transmit-receive unit 3, B mode processor 4, Doppler processor 5, image processor 6, display unit 7, contrast timer 8 and voice output unit 9 referred to above. The controller 10 supplies control signals to their respective parts to control their operations. Various signals are inputted to the controller 10 from the respective controlled parts. A B mode operation (including a harmonic B mode) and a Doppler mode operation are executed under the control of the controller 10.

The operation unit 11 is connected to the controller 10. The operation unit 11 is controlled by an operator to input suitable instructions and information to the controller 10. The operation unit 11 is provided with, for example, a keyboard, a pointing device and other operation devices.

The operation of the ultrasonic imaging apparatus will next be explained.

The ultrasonic probe 2 is brought into contact with a desired point of a subject with a contrast agent administered thereto. The operation unit 11 is operated to carry out an imaging operation utilizing, for example, a B mode and a Doppler mode in combination. Incidentally, the simply-described B mode includes a harmonic B mode for generating a harmonic image in addition to a normal B mode based on a basic-wave echo. Thus, B mode imaging and Doppler mode imaging are carried out on a time-sharing basis under the control of the controller 10. That is, for example, a mixed scan of the B mode and the Doppler mode is carried out at such a rate that the scan of the B mode is performed once for a predetermined number of times of the scan of the Doppler mode.

In the B mode, the transmit-receive unit 3 scans the interior of the subject 100 on a sound-rays sequential basis through the ultrasonic probe 2 and receives their echoes sequentially. The B mode processor 4 logarithmically amplifies an echo receive signal inputted from the transmit-receive unit 3 through the use of the logarithmic amplifying unit 41, and detects an envelope thereof through the use of the envelope detection unit 42 to obtain an A scope signal, thereby forming B-mode image data or harmonic image data set every sound rays, based on the signal.

The image processor 6 allows the input data memory 65 to store the B-mode image data or harmonic image data set every sound rays, inputted from the B mode processor 4. Thus, a sound-ray data space about the B-mode image data or harmonic image data is formed within the input data memory 65.

In the Doppler mode, the transmit-receive unit 3 scans the interior of the subject 100 on a sound-rays sequential basis through the ultrasonic probe 2 and receives their echoes sequentially. At this time, a plurality of times of transmission of ultrasounds and reception of echoes per sound ray are carried out.

The Doppler processor 5 effects quadrature detection on each echo receive signal through the use of the quadrature detection unit 51 and effects MTI processing thereof through the use of the MTI filter 52. The Doppler processor 5 determines auto-correlation with the auto-correlation calculating unit 53 and determines a flow velocity V from the result of auto-correlation calculation through the use of the average flow velocity calculating unit 54. Further, the Doppler processor 5 determines a dispersion T through the use of the dispersion calculating unit 55 and obtains power PW through the use of the power calculating unit 56. These calculated values respectively result in data indicative of the flow velocity, dispersion and power of an echo source every sound rays and pixels.

The image processor 6 allows the input data memory 65 to store the respective Doppler image data set for each sound ray and pixel, which are inputted from the Doppler processor 5. Thus, sound-ray data spaces about the respective Doppler image data are formed within the input data memory 65.

The CPU 60 scans and converts the B-mode image data, the harmonic image data and the respective Doppler image data of the input data memory 65 through the use of the DSC 66 and writes them into the image memory 67. At this time, the Doppler image data are respectively written as flow-velocity distribution image data utilizing the flow velocity V and the dispersion T in combination, power Doppler image data using the power PW or power Doppler image data with dispersion, utilizing the power PW and the dispersion T in combination, and dispersion image data using the dispersion T.

The CPU 60 writes the B-mode image data, harmonic image data and respective Doppler image data into discrete areas of the image memory 67. An image based on these B-mode image data, harmonic image data and respective Doppler image data is displayed on the display unit 7.

The B-mode image shows a tomogram of an in-vivo tissue on a sound-ray scanning plane. The harmonic image shows a tomogram using a secondary higher harmonic wave obtained from the contrast agent. An image corresponding to a region of interest over which the contrast agent is distributed, is also displayed in the B-mode image. Of color Doppler images, a flow velocity distribution image results in an image indicative of a two-dimensional distribution of a flow velocity of an echo source. In the present image, display colors are made different according to the direction of a flow thereof. The display colors are made different in luminance according to the flow velocity. The color-mixed amount of predetermined colors is enhanced according to the dispersion, whereby the purity of each display color is changed.

A power Doppler image results in an image indicative of a two-dimensional distribution of power of a Doppler signal. The location of the echo source exercised according to the image is shown. The luminance of each display color for the image corresponds to the power. When the power is utilized in combination with the dispersion, the color-mixed amount of predetermined colors is enhanced according to the dispersion to change the purity of each display color. A dispersion image results in an image indicative of a two-dimensional distribution of dispersed values. This image also indicates the location of a moving echo source. The luminance of each display color is associated with the magnitude of the dispersion.

When the above images are displayed on the display unit 7, the display memory 68 combines the images with the B-mode image or harmonic image, and the combined image is displayed on the display unit 7, whereby a color Doppler image evident in position relationship with an in-vivo tissue can be observed.

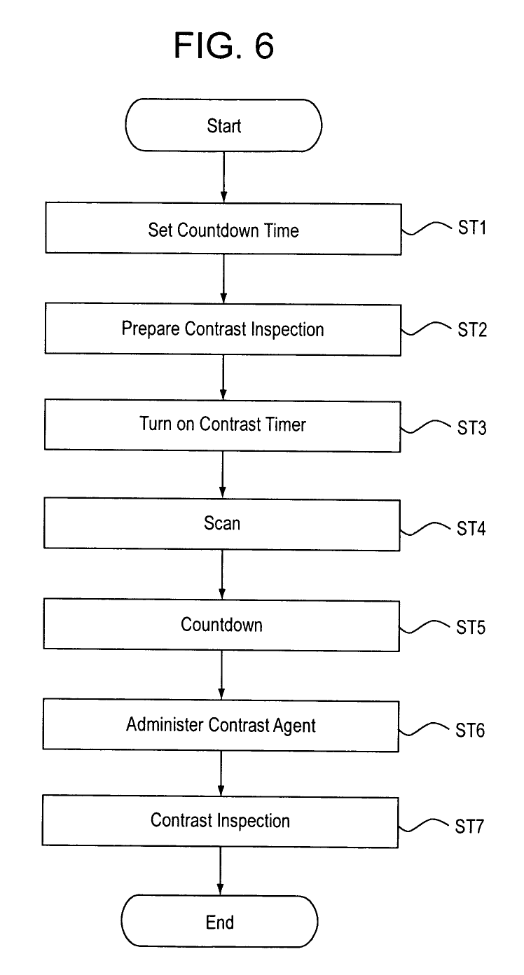

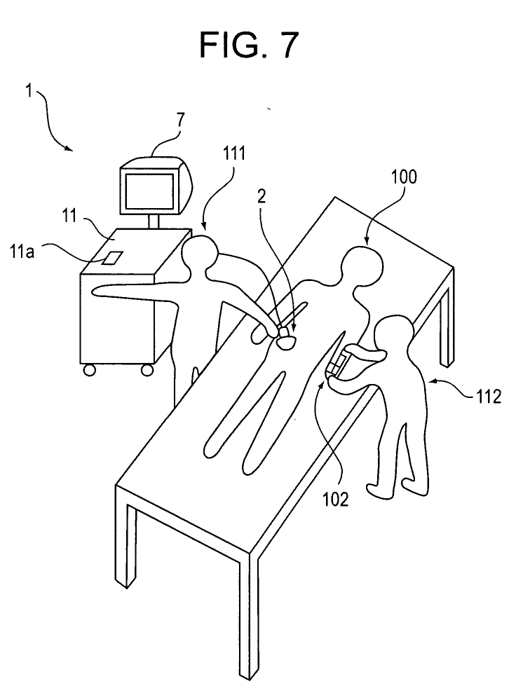

A method of performing a contrast inspection using the ultrasonic imaging apparatus 1 will next be described with reference to FIGS. 6 and 7. FIG. 6 is a flowchart showing a procedure for the contrast inspection, and FIG. 7 is a diagram showing the manner of the contrast inspection, respectively.

In the contrast inspection, the ultrasonic imaging apparatus 1 is placed on one side of a bed with a subject 100 placed thereon, for example, as shown in FIG. 7. An operation unit of the ultrasonic imaging apparatus 1 and an operator 111 who brings the ultrasonic probe 2 into contact with the subject 100, are located on one side thereof. An operator 112 who pushes an injector 102 for administering a contrast agent, is located on the other side of the bed.

In the contrast inspection, for example, the operator 111 first sets a countdown time via the operation unit 11 (Step ST1). Thus, the countdown time is set to about 10 seconds, for example.

Next, the contrast inspection is prepared (Step ST2). Upon preparation for the contrast inspection, as shown in FIG. 7, the injector 102 for injecting the contrast agent into the subject 100 is fixed and the operator 112 is placed in a state of being able to inject the contrast agent therein by simply pressing a piston of the injector 102. Further, the operator 111 brings the ultrasonic probe 2 into contact with the subject 100 and observes a tomogram displayed on the display unit 7 by scanning of ultrasounds to thereby confirm an imaged or photographed region.

Next, as shown in FIG. 7, the operator 111 presses an operation or control button 1 la of the contrast timer provided in the operation unit 11 (Step ST3). Consequently, the contrast timer 8 is turned on. Thereafter, the operator 111 scans the optimum section of the subject 100 (Step ST4).

On the ultrasonic imaging apparatus 1 side, the countdown made by the contrast timer 8 is notified from the voice output unit 9 via voice (Step ST5). The voice output unit 9 may be built in the display unit 7 shown in FIG. 7, or another voice output unit may be provided.

With the notification of the contrast start time by the voice output unit 9, the operator 112 presses the piston of the injector to administer the contrast agent into the subject 100 (Step ST6). At this time, the operator 111 is capable of concentrating on the scan for the subject 100 and the observation of the tomogram displayed on the display unit 7.

After the administration of the contrast agent, the contrast inspection for continuously performing an ultrasonic photography for a predetermined time interval is carried out (Step ST7). After the elapse of the predetermined time interval, the contrast timer 8 is deactivated and the scan is stopped, whereby the contrast inspection is completed.

In the ultrasonic imaging apparatus 1 according to the present embodiment as described above, when the contrast timer 8 is brought to an on state, the countdown up to the administration of the contrast agent is carried out and notified by the voice output unit 9 via voice.

Since the time when the button of the operation unit 11 is pressed to turn on the contrast timer and the time when the contrast agent is administered can be shifted from each other, the operator 111 is able to concentrate on the scan. Since the operator 112 may administer the contrast agent 101, based on the countdown notified by the voice output unit 9, the operator 112 is able to exactly administer the contrast agent at a predetermined time even though the operator 112 has no voice conversation with the operator 111.

Accordingly, a burden on the operator 111 at the time of administration of the contrast agent can be reduced and hence a stable contrast inspection can be carried out.

### Second Embodiment

The present embodiment will explain an example in which a means for notifying a countdown measured by a contrast timer is configured of a display unit 7 without using the voice output unit 9. It is not necessary to provide the voice output unit 9 shown in FIG. 1 in the present embodiment.

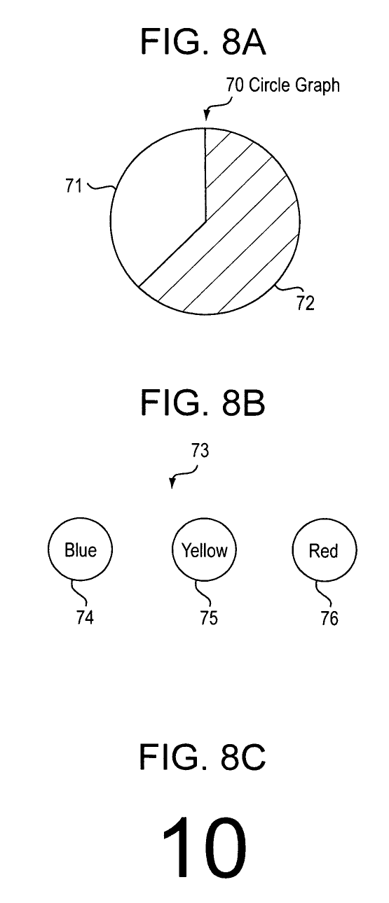

The display unit 7 is controlled by a controller 10 and displays a countdown measured by the contrast timer 8 on an area different from an area on which a tomogram is displayed. FIGS. 8(a) through 8(c) are diagrams showing countdown notified images by the display unit 7.

As shown in FIG. 8(a), for example, a circle graph 70 is displayed in the margin of the screen of the display unit 7. The circle graph 70 comprises two sections or parts 71 and 72 different in brightness. As a contrast-agent administration time presses, the area of the section 72 low in brightness is set so as to increase and the area of the section 71 high in brightness is set so as to decrease. Consequently, an operator 112 is able to recognize the contrast-agent administration time. Incidentally, hue may be used as an alternative to the brightness and a bar graph may be used in place of the circle graph.

The countdown notified images may be configured as three color signal display images 73 similar to traffic signals as shown in FIG. 8(b). The color signal display images 73 comprise a blue signal display image 74, a yellow signal display image 75 and a red signal display image 76. As the time up to the administration of the contrast agent becomes short, the blue signal display image 74, the yellow signal display image 75 and the red signal display image 76 are displayed in order. Incidentally, other images are assumed to be achromatic during a period in which one image is being displayed. By doing so, the operator 112 is able to recognize the contrast-agent administration time.

The countdown notified images may be displayed by numerals as shown in FIG. 8(c). In the present embodiment, the numerals change in order of 10, 9, 8, . . . 3, 2, 1, 0, for example. Even by doing so, the operator 112 is able to recognize the contrast-agent administration time.

Incidentally, although the countdown notified images have been displayed on the display unit 7 together with a tomogram, another display device for displaying the countdown notified images may be provided on the side close to the operator 112.

### Third Embodiment

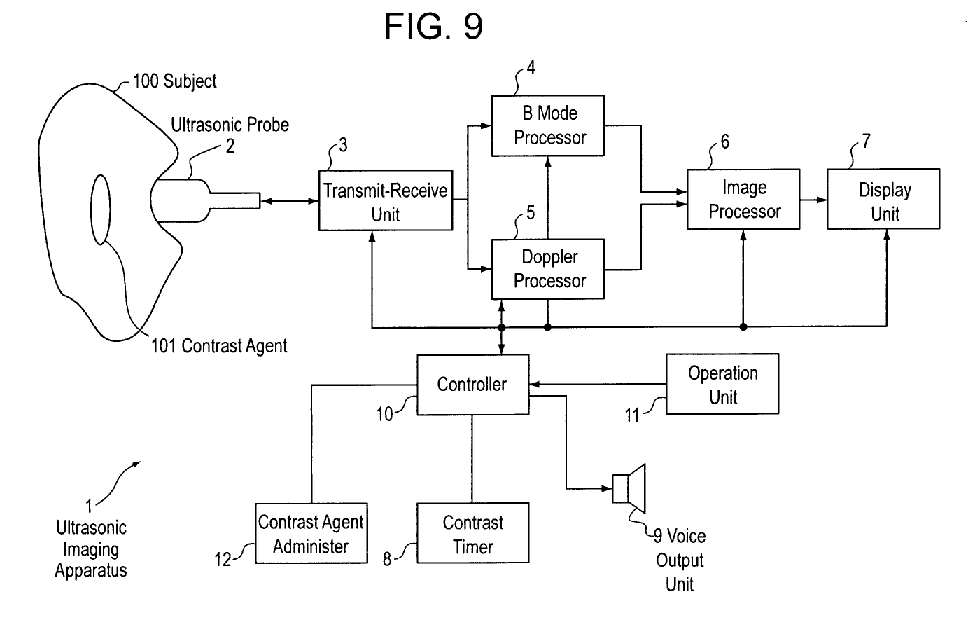

FIG. 9 is a block diagram showing one example of a configuration of an ultrasonic imaging apparatus according to the present embodiment. Incidentally, components similar to those shown in FIG. 1 are given the same reference numerals and their description will therefore be omitted.

In the present embodiment, a contrast agent administer 12 is used which automatically administers a contrast agent to a subject 100. The contrast agent administer 12 is normally made up of an enclosure or case different from a main body of the ultrasonic imaging apparatus 1. The contrast agent administer 12 drives a piston of an injector set to the subject 100 in accordance with a control signal supplied from a controller 10. Incidentally, the contrast agent administer is not linked to the ultrasonic imaging apparatus 1 in the prior art.

In the present embodiment, the contrast agent administer 12 is connected to the controller 10. When the time counted down by a contrast timer 8 reaches 0, i.e., a contrast-agent administration time, a control signal is outputted from the controller 10, so that the contrast agent administer 12 administers the contrast agent to the subject 100.

Although a voice output unit 9 does not need to be provided in the present embodiment, it may be provided to allow an operator 111 on the scanning side to recognize the contrast agent administration time. Incidentally, countdown notified images may be displayed on a display unit 7 as an alternative to the voice output unit 9.

Since the time when a button of an operation unit 11 is pressed to turn on the contrast timer and the time when the contrast agent is administered can be shifted from each other, the operator 111 is able to concentrate on the scan. Since the contrast agent is automatically administered by the contrast agent administer 12, the operator 111 can also perform a contrast inspection singly.

Thus, a burden on the operator 111 at the time of administration of the contrast agent can be reduced and hence a stable contrast inspection can be carried out.

### Fourth Embodiment

In the present embodiment, sound pressures of ultrasounds sent by a transmit-receive unit 3 are switched in the vicinity of the time when the administration of a contrast agent is started, under the control of a controller 10. These are applied to the first through third embodiments.

For example, an ultrasound high in sound pressure is transmitted by the transmit-receive unit 3 till the contrast-agent start time, whereas an ultrasound low in sound pressure is transmitted by the transmit-receive unit 3 after the contrast-agent start time. This is effective in the use of such a contrast agent that will break in the case of the ultrasound high in sound pressure. There is an advantage that since echoes reflected from a tissue of a subject are received before the administration of the contrast agent, the ultrasound high in sound pressure makes it possible to generate a more satisfactory B-mode image.

Alternatively, the ultrasound low in sound pressure is transmitted by the transmit-receive unit 3 till the contrast-agent start time, whereas the ultrasound high in sound pressure is transmitted by the transmit-receive unit 3 after the contrast-agent start time. This is effective in using such a contrast agent that received echoes cannot be obtained if it is not broken by the ultrasound high in sound pressure.

Many widely different embodiments of the invention may be configured without departing from the spirit and the scope of the present invention. It should be understood that the present invention is not limited to the specific embodiments described in the specification, except as defined in the appended claims.

1. An ultrasonic imaging apparatus comprising:
an ultrasonic transmit-receive device which scans an interior of a subject by ultrasounds and thereby receives echoes from the subject;
an image generating device which generates an image based on the received echoes;
a time measuring device which counts down a time from a pre-set time to the time when the administration of a contrast agent is started; and
a notifying device which notifies the time up to the contrast-agent administration start time, counted down by the time measuring device.

2. The ultrasonic imaging apparatus according to claim 1, further comprising an operation device,
wherein the time measuring device counts down a time from a pre-set time to a contrast-agent administration start time in response to an input given from the operation device.

3. The ultrasonic imaging apparatus according to claim 1, wherein the notifying device notifies a time up to the contrast-agent administration start time by voice.

4. The ultrasonic imaging apparatus according to claim 1, wherein the notifying device displays a time up to the contrast-agent administration start time.

5. The ultrasonic imaging apparatus according to claim 1, wherein the ultrasonic transmit-receive device switches sound pressures of the ultrasounds used to scan when the contrast-agent administration start time is reached.

6. The ultrasonic imaging apparatus according to claim 1, wherein the ultrasonic transmit-receive device performs switching between ultrasonic scan modes when the contrast-agent administration start time is reached.

7. The ultrasonic imaging apparatus according to claim 1, wherein the image generating device generates a B mode image or a harmonic image.

8. The ultrasonic imaging apparatus according to claim 1, wherein the image generating device generates a color Doppler image.

9. An ultrasonic imaging apparatus comprising:
an ultrasonic transmit-receive device which scans an interior of a subject by ultrasounds and thereby receives echoes from the subject;
an image generating device which generates an image based on the received echoes;
a time measuring device which counts down a time from a pre-set time to the time when the administration of a contrast agent is started; and
a contrast-agent administering device which administers a contrast agent to the subject, based on countdown information obtained by the time measuring device when the contrast-agent administration start time is reached.

10. The ultrasonic imaging apparatus according to claim 9, further comprising an operation device,
wherein the time measuring device counts down a time from a pre-set time to a contrast-agent administration start time in response to an input given from the operation device.

11. The ultrasonic imaging apparatus according to claim 9, further comprising a notifying device which notifies a time up to the contrast-agent administration start time, counted down by the time measuring device.

12. The ultrasonic imaging apparatus according to claim 11, wherein the notifying device notifies a time up to the contrast-agent administration start time by voice.

13. The ultrasonic imaging apparatus according to claim 11, wherein the notifying device displays the time up to the contrast-agent administration start time.

14. The ultrasonic imaging apparatus according to claim 9, wherein the ultrasonic transmit-receive device switches sound pressures of the ultrasounds used to scan when the contrast-agent administration start time is reached.

15. The ultrasonic imaging apparatus according to claim 9, wherein the ultrasonic transmit-receive device performs switching between ultrasonic scan modes when the contrast-agent administration start time is reached.

16. The ultrasonic imaging apparatus according to claim 9, wherein the image generating device generates a B mode image or a harmonic image.
